(19) United States
(12) Patent Application Publication
(10) Pub. No.: US 2004/0176637 A1
(43) Pub. Date: Sep. 9, 2004

(54) Process for preparation of 2-chlorophenylglycine derivatives and enantiomerically separation

(75) Inventors: Simon Shin-Shing Lin, Taipei (TW); Cheng-Chung Chen, Taipei (TW)

Correspondence Address: DYKAS, SHAVER & NIPPER, LLP, P.O. BOX 877, 802 WEST BANNOCK STREET, SUITE 405, BOISE, ID, 83701, US

(73) Assignee: Robert C. C. Wu, Taipei (TW)

(21) Appl. No.: 10/382,129

(22) Filed: Mar. 3, 2003

(51) Int. Cl. C07C 229/36

(52) U.S. Cl. 562/401; 562/443

(57) ABSTRACT

The present invention relates to novel processes and intermediates for the preparation of R(−)-α-2-chlorophenylglycine, S(+)-α-2-chlorophenylglycine, and RS-2-chlorophenylglycine derivatives of formulas I, II and III, respectively. The resulting enantioseparative system was validated in order to evaluate the presence of the enantiomer in pharmaceutical samples. These compounds are found useful as an active ingredient for the pharmaceutical intermediate or as an active ingredient as the tools for delivery of drugs.

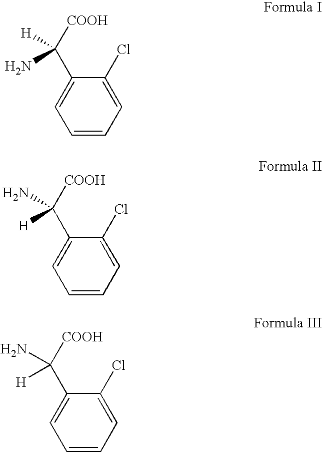

### BACKGROUND OF THE INVENTION

1. Field of the Invention

The invention relates to novel processes and intermediates for the preparation of R(−)-α-2-chlorophenylglycine, S(+)-α-2-chlorophenylglycine, and RS-2-chlorophenylglycine derivatives of the formula I, II and III which was synthesized and separated in several steps from 2-chlorophenylglycine and camphor sulfonic acid; for this purpose (+)-camphor-10-sulfonic acid, a chiral ion-pairing reagent, was found to be an essential additive in obtaining a baseline separation. The resulting enantioseparative system was validated in order to evaluate the presence of the enantiomer in pharmaceutical samples. These compounds are found useful as an active ingredient for the pharmaceutical synthesis of taxol, taxotere, clopidogrel, chlorfenapyr (Insecticide/Acaricide), AIDS and some antibiotics etc.

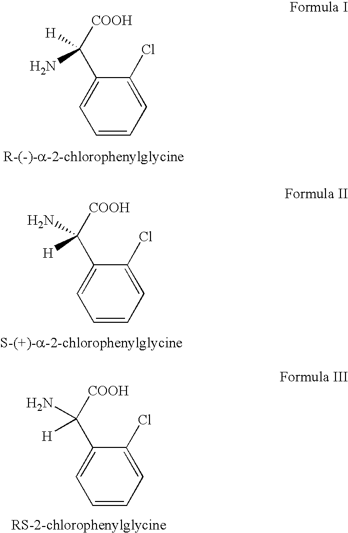

2. Description of the Prior Art

Suitable conditions for the enantioseparation were found by variation of the separation conditions. The influence of addition of organic solvents like acetonitrile or methanol, and other chiral additives (camphor-10-sulfonic acid, malic acid and tartaric acid) was examined. The addition of an organic modifier resulted in different effects on micelle formation, and thereby on the separation. The used chiral additives did not improve the selectivity. Salami, M. Electrophoresis, vol. 22, pp. 3291-3296 (2001). In the present work the behavior of the compounds in preparation systems containing camphor sulfonic acid has been studied. While, in the synthesis of S-(+)-2-chlorophenylglycine indicating the camphor sulfonic acid was observed, which caused significant increase of yield in the preparation systems with mother liquid as re-crystallization and recycle process. This phenomenon is interpreted by the formation of (+)-2-chlorophenylglycine/d-camphor sulfonic acid salt on the mother liquid layer and as a consequence of mixed isomeric mixtures mechanism. Theoretical considerations as well as the identical separation of the mixture make plausible the appearance and separation of conformational enantiomers.

The S-(+)-2-chlorophenylglycine was synthesized, and its enantiomers were obtained on a multigram scale in >99% optical purity by optical resolution of the racemate with the camphor sulfonic acid. The absolute configuration of S-(+)-2-chlorophenylglycine was determined by analysis of the salt with (+)-camphoric sulfonic acid. Since the chirality of the starting material was known, and the relative configuration of compounds were obtained by analysis, the assignment of the absolute stereochemistry of the entire series could be made.

The selectivity of separation of the 2-chlorophenylglycine enantiomers increase with rising concentration of methanol in aqueous-organic mobile phases, pH and temperature. The retention behavior of the enantiomers of underivatized phenylglycine was studied on a Chirobiotic T column packed with amphoteric glycopeptide teicoplanin covalently bonded to the surface of silica gel. The band profiles of the less retained L-phenylglycine are symmetrical, but the band profiles of the more strongly retained D-phenylglycine are tailing in all mobile phases tested. The band broadening does not diminish even at very low concentrations of phenylglycine, so that it cannot be attributed to possible column overload. The analysis of the band profile using the stochastic theory of chromatography suggests that the broadening can be attributed to at least two additional chiral centers of adsorption in the stationary phase contributing to the retention of the more strongly retained enantiomer in addition to the adsorption of the less retained one. This behavior can be explained by the complex structure of the teicoplanin chiral stationary phase. Jander P, Backovska V, Felinger A. J. Chromatogr. A 2001 Jun. 1;919(1):67-77.

### SUMMARY OF THE INVENTION

In this invention, we prepared and separated various racemic 2-chlorophenylglycine containing S-(+)-2-chlorophenylglycine, R-(−)-2-chlorophenylglycine and RS-2-chlorophenylglycine, respectively. A series of racemic 2-chlorophenylglycine having the following formula are synthesized and separated in the present invention:

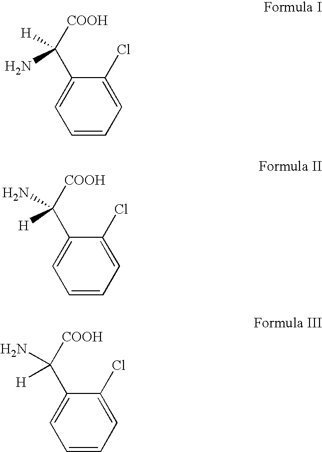

wherein configurational form of isomers are S-(+), R-(−) and racemate. Preferably, configurational form of isomers of 2-chlorophenylglycine of the formula I, II and III are enantiomers. Current work details on-going efforts to improve the effectiveness of this type of enantiomers. The analytes used in this study included various phenylglycine homologues. In an attempt to increase enantioselectivity, the effect of solvent, temperature and pH modifiers was evaluated in an aqueous mobile phase containing sulfuric acid.

S-form of 2-chlorophenylglycine having the following formula was synthesized and separated by the present invention. R-form of 2-chlorophenylglycine having the following formula was synthesized and separated by the present invention. Enantiomers of 2-chlorophenylglycine having the following formula was synthesized and separated by the present invention.

### BRIEF DESCRIPTION OF THE DRAWINGS

FIG. 1 (Scheme 1) shows synthetic method of 2-Chlorophenylglycine.

FIG. 2 (Scheme 2) shows resolution procedures on racemic mixture of 2-chlorophenylglycine.

Table. 1 shows the ¹H NMR, IR of S-form of 2-chlorophenylglycine.

### DETAILED DESCRIPTION OF THE INVENTION

In this invention, we prepared and separated various racemic 2-chlorophenylglycine containing S-(+)-2-chlorophenylglycine, R-(−)-2-chlorophenylglycine and RS-2-chlorophenylglycine, respectively. A series of racemic 2-chlorophenylglycine having the following formula are synthesized and separated in the present invention:

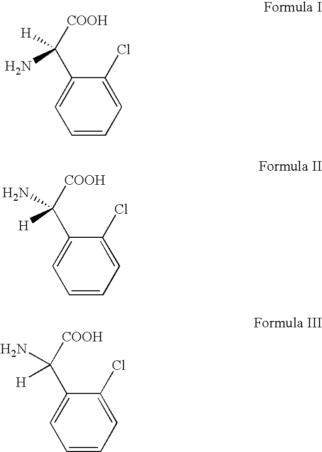

R(−)-α-2-chlorophenylglycine S(+)-α-2-chlorophenylglycine RS-2-chlorophenylglycine wherein configurational form of isomers are S-(+), R-(−) and racemate. Preferably, configurational form of isomers of 2-chlorophenylglycine of the formula I, II and III are enantiomers. Current work details on-going efforts to improve the effectiveness of this type of enantiomers. The analytes used in this study included various phenylglycine homologues. In an attempt to increase enantioselectivity, the effect of solvent, temperature and pH modifiers was evaluated in an aqueous mobile phase containing sulfuric acid.

S-form of 2-chlorophenylglycine having the following formula was synthesized and separated by the present invention. R-form of 2-chlorophenylglycine having the following formula was synthesized and separated by the present invention. Enantiomers of 2-chlorophenylglycine having the following formula was synthesized and separated by the present invention.

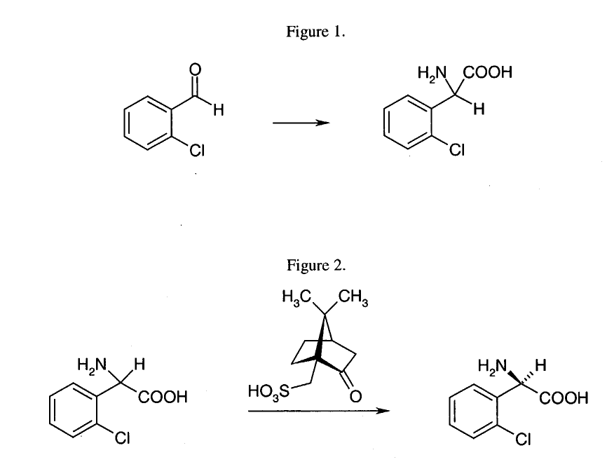

The 2-chlorophenylglycine analogs were synthesized according to the experiment as below. The enantiomerically pure S-form of 2-chlorophenylglycine, R-form of 2-chlorophenylglycine and racemic 2-chlorophenylglycine were determined by ¹H-NMR spectroscopy and enantioselectivities were determined by optical rotation. The enantiomeric excesses are dependent on the reaction temperature, concentration, pH and solvent when the reaction were carried out with the complex prepared from 2-Chlorobenzaldehyde, ammonium hydrogencarbonate, and sodium cyanide. The high asymmetric induction in the reaction can be rationalized by assuming that the reaction proceeds via the intermediacy of enantiomer shown in Scheme. The enantiomeric purity of the stereoisomers was determined by a high-performance liquid chromatography-chiral stationary phase technique (HPLC-CSP). Accordingly, in one embodiment of the invention, there is provided an compound according to formula I, II, III as defined below and shown in FIG. 1, said compound containing enantiomeric purity of the stereoisomers.

2-Chlorophenylglycine was prepared according to a synthetic method depicted in FIG. 1. A solution of 2-chlorobenzaldehyde, ammonium hydrogencarbonate (NH₄HCO₃; 23.7 g), and sodium cyanide (NaCN; 14.7 g) in 500 ml of methanol and 500 ml water, was stirred at 65-70° C. for 5 h. The solution was concentrated and transferred to autoclave and added 45% NaOH solution, was refluxed for 4 h at 120° C. The reaction mixture was added 2 g of active carbon and stir for 10 min. The active carbon was filtered and the pH of filtrate was adjusted by 50% H₂SO₄ to 7-8. The precipitated was filtrated and washed with water and gave 27 g (58%) of 2-chlorophenylglycine. RS-2-chlorophenylglycine specific rotation.

A racemic mixture of 2-chlorophenylglycine was prepared according to general synthetic procedures. The two enantiomers of 2-chlorophenylglycine were resolved by D-camphor sulfonic acid in water. A solution of racemic 2-chlorophenylglycine (60 g) and D-camphor sulfonic acid (80 g) in 360 ml of water, was stirred at 85° C. for 30 min. The precipitate S(+)CPG-DSC was filtered and washed with water. The filtrate was mother liquid as recycle. The wet S(+)CPG-DSC was dissolved in 80 ml of water and adjusted to pH 7. The precipitate was filtered and washed with water, dried to give 12.3 g of S(+)-2-chlorophenylglycine. The filtrate was added to mother liquid and washed with 45% NaOH and concentrated and then added 70 ml of HCl to regenerate the target compound. S(+)-2-Chloro-Phenylglycine, R(−)-2-Chloro-Phenylglycine, and RS-2-Chloro-Phenylglycine were obtained.

The characterized of prepared process was found to be an essential additive in obtaining a baseline separation, whether pH 7-8 adjustment using NaOH solution and H₂SO₄ solution. The color removing using active carbon, and that the chiral ion-pairing reagent was D-camphor sulfonic acid.

A pharmaceutical composition for the synthesis of taxol, taxotere, clopidogrel, chlorfenapyr (Insecticide/Acaricide), AIDS and some antibiotics or a pharmaceutical acceptable material thereof, S-(+)-2-chlorophenylglycine and R-(−)-2-chlorophenylglycine as an active ingredient in preparation for a pharmaceutically acceptable carrier or the active ingredient.

The design and synthesis of novel chiral catalysts for asymmetric reactions continues to be an important and active area of research. While the invention is susceptible of various modifications and alternative constructions, certain illustrated embodiments thereof have been shown in the drawings and will be described below in detail. It should be understood, however, that there is no intention to limit the invention to the specific form disclosed, but, on the contrary, the invention is to cover all modifications, alternative constructions, and equivalents falling within the spirit and scope of the invention as defined in the claims.

### Detailed Description of Experiment

Materials 2-Chlorobenzaldehyde, ammonium hydrogencarbonate (NH₄HCO₃), sodium cyanide, NaOH, H₂SO₄ and methanol were commercial materials. Melting points were determined with a Büchi B-545 melting point apparatus and are uncorrected. All reactions were monitored by TLC (silica gel 60 F₂₅₄). ¹H-NMR: Varian GEMINI-300 (300 MHz) and Brucker AM-500 (500 MHz); δ values are in ppm relative to TMS as an internal standard. Fourier-transform IR spectra (KBr): Perkin-Elmer 983G spectrometer. The UV spectra were recorded on a Shimadzu UV-160A. Typical experiments illustrating the general procedures for the preparation are described below.

### EXAMPLE 1

### General Synthetic Procedures

2-Chlorophenylglycine was prepared according to a synthetic method depicted in Scheme 1. A solution of 2-chlorobenzaldehyde, ammonium hydrogencarbonate (NH₄HCO₃; 23.7 g), and sodium cyanide (NaCN; 14.7 g) in 500 ml of methanol and 500 ml water, was stirred at 65-70° C. for 5 h. The solution was concentrated and transferred to autoclave and added 45% NaOH solution, was refluxed for 4 h at 120° C. The reaction mixture was added 2 g of active carbon and stir for 10 min. The active carbon was filtered and the pH of filtrate was adjusted by 50% H₂SO₄ to 7-8. The precipitated was filtrated and washed with water and gave 27 g (58%) of 2-chlorophenylglycine. RS-2-chlorophenylglycine specific rotation +0.16 (C=1, 1N HCl); mp 185.4-186.8° C.

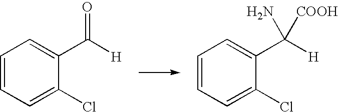

### EXAMPLE 2

### General Resolution Procedures

A racemic mixture of 2-chlorophenylglycine was prepared according to general synthetic procedures. The two enantiomers of 2-chlorophenylglycine were resolved by D-camphor sulfonic acid in water. A solution of racemic 2-chlorophenylglycine (60 g) and D-camphor sulfonic acid (80 g) in 360 ml of water, was stirred at 85° C. for 30 min. The precipitate S(+)CPG-DSC was filtered and washed with water. The filtrate was mother liquid as recycle. The wet S(+)CPG-DSC was dissolved in 80 ml of water and adjusted to pH 7. The precipitate was filtered and washed with water, dried to give 12.3 g of S(+)-2-chlorophenylglycine. [α]=+115.6°, C=1N HCl. The filtrate was added to mother liquid and washed with 45% NaOH and concentrated and then added 70 ml of HCl to regenerate the target compound. S(+)-2-Chloro-Phenylglycine: specific rotation +114.9 (C=1, 1N HCl); mp 184.4-185.7° C. R(−)-2-Chloro-Phenylglycine: specific rotation −111.5 (C=1, 1N HCl); mp 184.9-185.8° C. RS-2-Chloro-Phenylglycine: specific rotation +0.16 (C=1, 1N HCl); mp 185.4-186.8° C.

While there is shown and described the present preferred embodiment of the invention, it is to be distinctly understood that this invention is not limited thereto but may be variously embodied to practice within the scope of the following claims. From the foregoing description, it will be apparent that various changes may be made without departing from the spirit and scope of the invention as defined by the following claims.

TABLE 1
The ^{1H NMR}, IR of S-form of 2-chlorophenylglycine.
S-(+)-2-chlorophenylglycine

| | | | | | |
|---|---|---|---|---|---|
| ¹H NMR | 7.456 | 7.437 | 7.429 | 7.423 | 7.367 |
| | 7.357 | 7.346 | 7.338 | 7.330 | 7.324 |
| | 7.317 | 7.311 | 7.297 | 7.291 | 5.074 |
| IR | 2363.63 | 1676.19 | 1633.33 | 1504.76 | 1376.19 |
| | 1047.61 | 747.61 | | | |

R-(−)-2-chlorophenylglycine

| | | | | | |
|---|---|---|---|---|---|
| ¹H NMR | 7.452 | 7.434 | 7.426 | 7.421 | 7.364 |
| | 7.353 | 7.343 | 7.335 | 7.327 | 7.320 |
| | 7.314 | 7.308 | 7.294 | 7.287 | 5.071 |
| IR | 2354.06 | 1680.95 | 1633.33 | 1504.76 | 1376.19 |
| | 747.61 | | | | |

RS-2-chlorophenylglycine

| | | | | | |
|---|---|---|---|---|---|
| ¹H NMR | 7.458 | 7.439 | 7.431 | 7.424 | 7.369 |
| | 7.360 | 7.348 | 7.340 | 7.332 | 7.326 |
| | 7.319 | 7.313 | 7.305 | 7.298 | 7.292 |
| | 5.077 | | | | |
| IR | 3052.63 | 2985.64 | 2612.44 | 2354.06 | 1642.85 |
| | 1576.19 | 1533.33 | 1438.09 | 1430.09 | 1347.61 |
| | 1190.47 | 1057.14 | 742.85 | | |

We claim:

1. Process for preparing the 2-chlorophenylglycine isomer of the compounds of formula 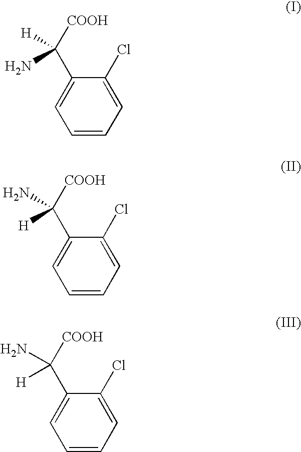
characterized in that a (+)-camphor-10-sulfonic acid, a chiral ion-pairing reagent, was found to be an essential additive in obtaining a baseline separation and recycling from the mother liquid, thereof, the compounds of formula of 2-chlorophenylglycine isomer wherein steric isomers are R(−)-α-2-chlorophenylglycine (I) and S(+)-α-2-chlorophenylglycine (II).
Wherein 2-chlorobenzaldehyde, ammonium hydrogencarbonate, and sodium cyanide in methanol and water was reacted.

2. Process for resolution of the enantiomeric isomer of the R(−)-α-2-chlorophenylglycine, S(+)-α-2-chlorophenylglycine from RS-2-chlorophenylglycine.

3. A racemic mixture of 2-chlorophenylglycine was prepared according to general synthetic procedures. The two enantiomers of 2-chlorophenylglycine were resolved by D-camphor sulfonic acid in water. A solution of racemic 2-chlorophenylglycine and D-camphor sulfonic acid in water, was stirred at 85° C. for 30 min. The precipitate S(+)CPG-DSC(S(+)-chlorophenylglycine-d-camphoric sulfonic acid) was filtered and washed with water. The filtrate was mother liquid as recycle. The wet S(+)CPG-DSC was dissolved in water and adjusted to pH 7. The precipitate was filtered and washed with water, dried to give S(+)-2-chlorophenylglycine. The filtrate was added to mother liquid and washed with 45% NaOH and concentrated and then added HCl to regenerate the target compound.

4. Process according to claim 1, characterized in that the starting materials are 2-chlorobenzaldehyde, ammonium hydrogencarbonate, and sodium cyanide.

5. Process according to claim 1, characterized in that the pH adjustment using NaOH solution and H₂SO₄ solution.

6. Process according to claim 1, characterized in that the removing the color using active carbon.

7. Process according to claim 1, characterized in that the chiral ion-pairing reagent was D-camphor sulfonic acid.

8. Process according to claim 2, characterized in that the final resolution isomer is S(+)-α-2-chlorophenylglycine.

9. Process according to claim 2, characterized in that from the mother liquid resolution isomer is R(−)-α-2-chlorophenylglycine.

10. Process according to claim 2, characterized in that the cyclization is carried out in the presence of pH, temperature and water.

11. Process according to claim 2, characterized in that the chiral ion-pairing reagent was D-camphor sulfonic acid.

12. Enantiomeric isomer of the R(−)-α-2-chlorophenylglycine and S(+)-α-2-chlorophenylglycine and the racemic form of RS-2-chlorophenylglycine thereof.

13. A pharmaceutical composition for the synthesis of taxol, taxotere, clopidogrel, chlorfenapyr (Insecticide/Acaricide), AIDS and some antibiotics or a pharmaceutical acceptable material thereof, as an active ingredient in preparation for a pharmaceutically acceptable carrier or the active ingredient.
